(19) United States
(12) Patent Application Publication
(10) Pub. No.: US 2019/0070109 A1
(43) Pub. Date: Mar. 7, 2019

(54) PHARMACEUTICAL COMPOSITION FOR ORAL DELIVERY

(71) Applicant: National Tsing Hua University, Hsinchu (TW)

(72) Inventors: Hsing-Wen SUNG, Hsinchu (TW); Po-Yen LIN, Hsinchu (TW); Kuan-Hung CHEN, Hsinchu (TW)

(21) Appl. No.: 16/178,041

(22) Filed: Nov. 1, 2018

Related U.S. Application Data
(63) Continuation-in-part of application No. 15/797,413, filed on Oct. 30, 2017.

(30) Foreign Application Priority Data
Nov. 18, 2016 (TW) 105137833

(51) Int. Cl. A61K 9/00; A61K 9/107; A61K 31/337; A61K 31/12; A61K 47/12; A61K 9/48

(52) U.S. Cl. CPC A61K 9/0065; A61K 9/0053; A61K 9/107; A61K 9/4891; A61K 31/12; A61K 47/12; A61K 31/337

(57) ABSTRACT

A pharmaceutical composition for oral delivery is provided with a poorly water-soluble drug; solvent with lipophilic tails and hydrophilic ends; an acid initiator; and a foaming agent generating carbon dioxide bubbles when the acid initiator is dissolved into the intestinal fluid to form an acidic environment. The poorly water-soluble drug is dissolved in the solvent to form a self-assembled monolayer carrier system with the bile salts surrounding the carbon dioxide bubbles in water when the pharmaceutical composition is dissolved in an intestinal tract, and lipid oil drops containing the poorly water-soluble drug form when the carbon dioxide bubbles burst at the air-liquid interface in the intestinal tract.

### BACKGROUND OF THE INVENTION

### 1. Field of the Invention

The present invention relates to an oral chemotherapy platform, particularly to a pharmaceutical composition for oral delivery to form lipid oil drops as nano-carriers.

### 2. Description of the Prior Art

Many common hydrophobic drugs, such as curcumin, paclitaxel and doxorubicin, have been proved to have a good therapeutic effect in experiments. However, the hydrophobicity thereof hinders them from mixing homogeneously in fabrication, or makes them hard to disperse while they disintegrate in the digestive organs, or causes them to deposit. Thus, the hydrophobic drugs are hard to be absorbed by living bodies and suffer low bioavailability. The abovementioned problems may affect the therapeutic effect, generate some side-effects, retard extensive clinical application, and impede further development of the hydrophobic drugs. Therefore, hydrophobic drugs are normally administrated in intravenous infusion. In order to avoid the inconvenience of invasive treatment, the current tendency is to develop appropriate carriers for fabricating oral hydrophobic drugs.

The common carriers for oral drugs include liposomes, nanoparticle carriers made of chitosan and γ-polyglutamic acid (γ-PGA), etc. The chitosan and γ-PGA carrier system is characterized in good gastric acid tolerance and dissolvable in the small intestine to release active ingredients. However, the fabrication process of the drugs using the chitosan and γ-PGA carrier system is very complicated and unfavorable for mass production, wherein the ingredients of the drug are mixed and dried in a special process and then enveloped in gelatin capsules. The dissolution of a capsule in the small intestine is usually incomplete and hard to control, which is likely to degrade the effect of drugs. Therefore, an improved carrier of oral hydrophobic drugs should favor the users thereof.

### SUMMARY OF THE INVENTION

A pharmaceutical composition for oral delivery is provided to form self-emulsified lipid oil drops as nano-carriers in intestinal aqueous environment of living body. The pharmaceutical composition as an oral chemotherapy platform is based on a bubble-carrier system in intestinal aqueous environment of a living body, which includes a poorly water-soluble drug; a lipophilic or amphiphilic solvent; an acid initiator; and an foaming agent producing carbon dioxide bubbles when the acid initiator is dissolved in the intestinal fluid to form an acidic environment, wherein the poorly water-soluble drug attaches to the solvent to form a self-assembled monolayer carrier system with a bile salts surrounding the carbon dioxide bubbles in water when the pharmaceutical composition is dispersed in an intestinal tract, and lipid oil drops containing the poorly water-soluble drug form when the carbon dioxide bubbles burst at the air-liquid interface in the intestinal tract.

In one embodiment, the solvent comprises lipophilic fatty acids, phospholipid, triglyceride, lipid derivatives, or ester derivatives.

In one embodiment, the foaming agent comprises carbonates or bicarbonates.

In one embodiment, the acid initiator comprises diethylenetriaminepentaacetic dianhydride (DTPA anhydride), citric acid, organic acid anhydrides, or organic acids.

In one embodiment, the poorly water-soluble drug comprises curcumin, paclitaxel, doxorubicin, or their derivatives.

In one embodiment, the pharmaceutical composition for oral delivery is in form of a tablet or a capsule.

In one embodiment, the pharmaceutical composition for oral delivery further comprises an enteric coating enveloping the tablet or capsule.

In one embodiment, the enteric coating comprises a methacrylic acid copolymer, hypromellose phthalate, hydroxypropyl cellulose acetate, hydroxypropyl cellulose succinate, or carboxymethyl ethyl cellulose.

In one embodiment, on the condition of a weight of the poorly water-soluble drug in the range of 1-3 milligram, the lipophilic or amphiphilic solvent is in the range of 15-60 milligram, the acid initiator is in the range of 2-25 milligram, and the foaming agent is in the range of 1-20 milligram.

Below, embodiments are described in detail in cooperation with the attached drawings to make easily understood the objectives, technical contents, characteristics and accomplishments of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an ultrasonic image showing an interface of liquid and air according to the present invention.

FIG. 2A is a fluorescent image captured by a confocal microscope to show bubble carriers in water according to the present invention.

FIG. 2B is a diagram schematically illustrating a monolayer of the solvent molecules, poorly water-soluble drug and carbon dioxide bubbles according to the present invention.

FIG. 3A is a fluorescent image captured by a confocal microscope to show bubble carriers on water according to the present invention.

FIG. 3B is a diagram schematically illustrating double-layer nano-assemblies of solvent molecules, poorly water-soluble drug and carbon dioxide bubbles according to the present invention.

FIG. 4 shows the results of the drug release experiments of a poorly water-soluble drug in different dosage forms.

FIG. 5 shows the distributions of poorly water-soluble drug in different dosage forms in tissues of living bodies in different groups.

### DESCRIPTION OF THE PREFERRED EMBODIMENTS

The present invention will be described in detail with embodiments and attached drawings below. However, these embodiments are only to exemplify the present invention but not to limit the scope of the present invention. In addition to the embodiments described in the specification, the present invention also applies to other embodiments. Further, any modification, variation, or substitution, which can be easily made by the persons skilled in that art according to the embodiment of the present invention, is to be also included within the scope of the present invention, which is based on the claims stated below. Although many special details are provided herein to make the readers more fully understand the present invention, the present invention can still be practiced under a condition that these special details are partially or completely omitted. Besides, the elements or steps, which are well known by the persons skilled in the art, are not described herein lest the present invention be limited unnecessarily. Similar or identical elements are denoted with similar or identical symbols in the drawings. It should be noted: the drawings are only to depict the present invention schematically but not to show the real dimensions or quantities of the present invention. Besides, matterless details are not necessarily depicted in the drawings to achieve conciseness of the drawings.

A pharmaceutical composition is provided to form self-emulsified lipid oil drops as bubble-carrier for oral delivery, which is a mixture of a poorly water-soluble drug, a lipophilic or amphiphilic solvent, an acid initiator and a foaming agent. Next, the pharmaceutical composition may be in a gelatin capsule that is then coated with an enteric polymer. The solvent may include lipophilic fatty acids, phospholipid, triglyceride, lipid derivatives, or ester derivatives, and in one embodiment, the solvent is capric acid. The foaming agent may include carbonates or bicarbonates. The acid initiator may include organic acids or organic anhydrides. The acid initiator may be selected from a group including tartaric acid, malic acid, maleic acid, fumaric acid, succinic acid, lactic acid, ascorbic acid, amino acid, glycolic acid, adipic acid, citric acid, diethylenetriaminepentaacetic dianhydride (DTPA anhydride), citric acid anhydride, succinic acid anhydride, and combinations thereof. In one embodiment, the acid initiator is critic acid. The foaming agent, for example but not limited, is sodium bicarbonate. It is noted that citric acid and sodium bicarbonate may rapidly react with each other in water to produce carbon dioxide of gas bubbles that are present in soda at a high pressure. Furthermore, capric acid is a lipid-based fatty acid oil to be deprotonated upon exposure to water and acts as a solvent for poorly water-soluble drug. In one example of the present invention, the pharmaceutical composition includes various weights as follows: the poorly water-soluble drug of paclitaxel of 1-3 (+/−30%) mg; the solvent of capric acid of 15-60 (+/−10%) mg; the acid initiator of citric acid of 2-25 (+/−15%) mg and the foaming agent of sodium bicarbonate of 1-20 (+/−15%) mg.

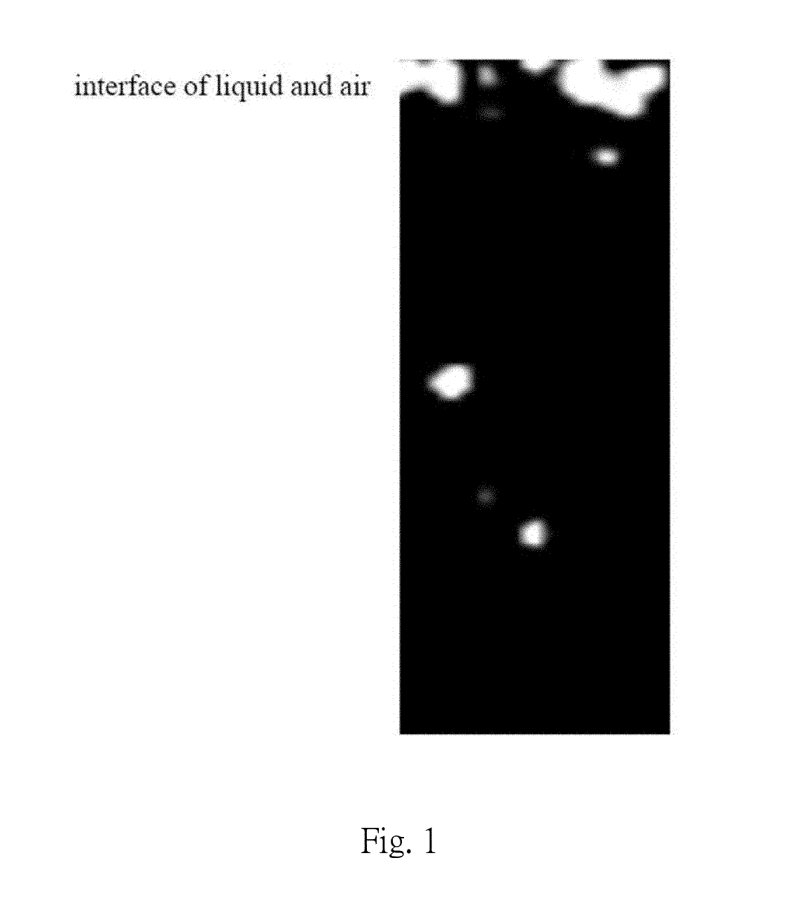

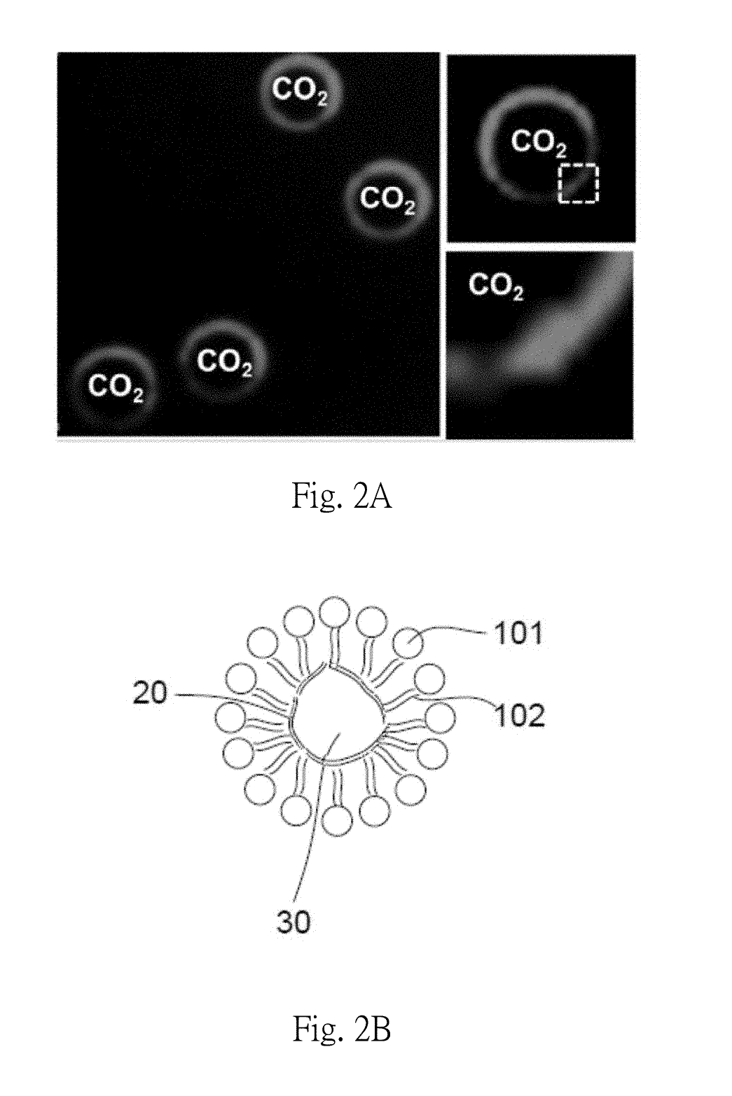

The pharmaceutical composition of the enteric-coated capsule of the present invention performs oral administration and dissolution in small intestine of a living body that is also an aqueous environment. While the pharmaceutical composition is exposed to the aqueous environment in an intestinal tract, the acid initiator is dissolved in the intestinal fluid to form an acidic environment in which the foaming agent of sodium bicarbonate decomposes to produce carbon dioxide bubbles. An interface of liquid and air may be seen by an ultrasonic image like one in FIG. 1. Next, please refer to FIG. 2A and FIG. 2B, these carbon dioxide bubbles 30 may be surrounded, and so stabilized by a monolayer of the amphiphilic bile salts with the nanofilm of solvent molecules (capric acid) dissolving the pharmaceutical composition anchored to the hydrophilic ends 101 of the amphiphilic bile salts. It is noted that the bile salts are derived from in small intestine of a living body, such as intrinsic amphiphilic bile salts or their derivatives. Shown in FIG. 2A and FIG. 2B, the lipophilic ends 102 of the bile salts surround one carbon dioxide bubble 30, and the nanofilm 20 that includes the poorly water-soluble drug dissolved in the solvent is anchored to form the self-assembled monolayer carrier system.

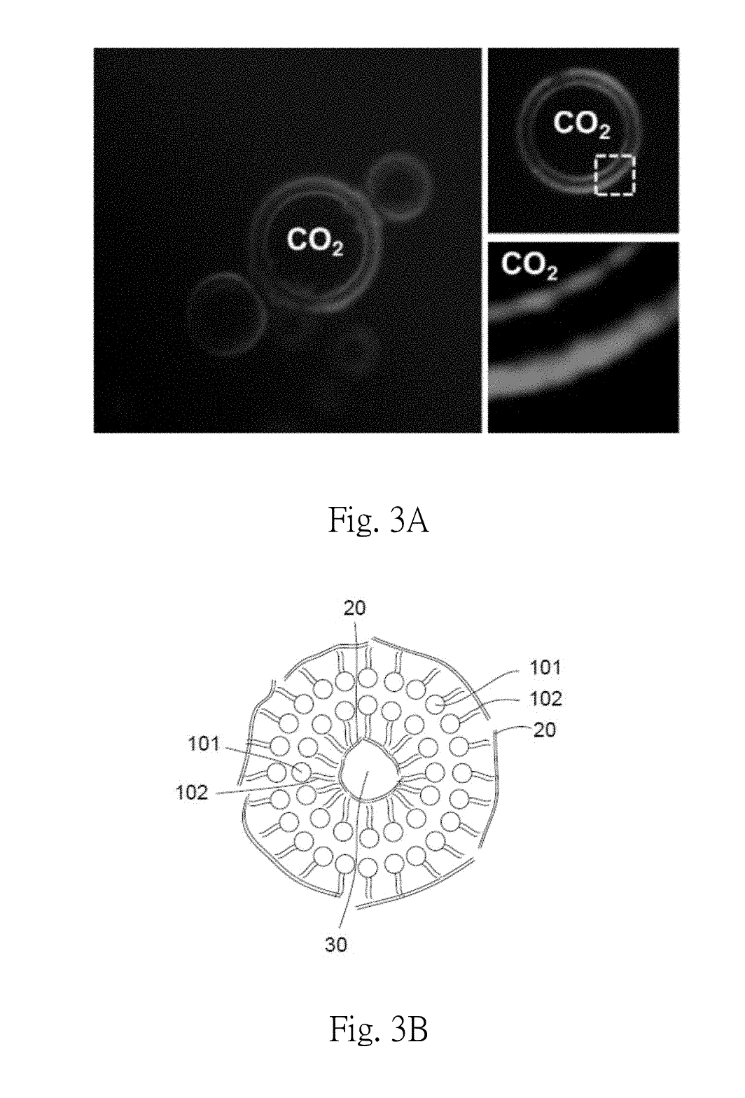

Next, the carbon dioxide bubbles 30 expand, rise and approach the interface of intestinal lumen, the self-assembled monolayer carrier system is transformed into double-layer nano-assemblies like ones in FIG. 3A and FIG. 3B. Shown in FIG. 3A and FIG. 3B, the hydrophilic ends 101 of the bile salts and the hydrophilic ends 101 of the self-assembled monolayer carrier system move toward each other and attract mutually to form double-layer nano-assemblies. Besides, the nanofilm 20 is anchored to the lipophilic tails 102 of the bile salts that moves toward the self-assembled monolayer carrier system. After the carbon dioxide bubbles 30 of the double-layer nano-assemblies burst at the interface of liquid and lumen, the solvent molecules (capric acid) and poorly water-soluble drug such as paclitaxel or curcumin and like are converted into oil-structured nano-emulsions via self-emulsification. Such the oil-structured nano-emulsions are viewed as self-emulsified drug-loaded lipid oil drops. Furthermore, the self-emulsified drug-loaded lipid oil drops are then internalized by M cells, most of which are located in Peyer's patches, and ultimately accumulated in pancreatic tumors via intestinal lymphatic transport.

Accordingly, the formation of carbon dioxide bubbles generates forces that promote the efficiency of dispersion of lipophilic solvent molecules with paclitaxel or curcumin and thus aggregation is prevented. At the bursting of the bubbles, the mechanical forces rip the double-layer nano-assemblies into oil-structured nano-emulsions. The encapsulation of paclitaxel or curcumin and like molecules in the lipid oil drops that are self-emulsified in the intestinal environment is a very important factor for their stabilization and absorption.

It is noted that the pharmaceutical composition for oral delivery that may form the self-emulsified lipid oil drops as nano-carriers of the present invention may be fabricated into tablets, capsules, or other oral dosage forms. Besides, the enteric coating may include a methacrylic acid copolymer, hypromellose phthalate, hydroxypropyl cellulose acetate, hydroxypropyl cellulose succinate, or carboxymethyl ethyl cellulose. While the self-emulsified lipid oil drops as nano-carriers for oral delivery is swallowed by a living body, the enteric coating can protect the pharmaceutical composition for oral delivery against the attack of gastric acid in the stomach. After entering the small intestine, the enteric coating of the pharmaceutical composition is dissolved. Moreover, pharmaceutical composition for oral delivery of the present invention may also include excipients, carriers, diluents, flavors, sweeteners, preservatives, antioxidants, humectants, buffer agents, release-control components, dyes, adhesives, suspending agents, dispersants, coloring agents, disintegrating agents, film forming agents, lubricants, plasticizers, edible oils, or combinations thereof.

Accordingly, the pharmaceutical composition for oral delivery that may form self-emulsified lipid oil drops as nano-carriers of the present invention is applied to transport a poorly water-soluble drug inside a living body. The hydrophobicity makes the poorly water-soluble drug hard to be dispersed uniformly inside a living body and thus hard to be absorbed by the living body, causing a problem of low bioavailability. In one embodiment, the poorly water-soluble drug includes curcumin, paclitaxel, doxorubicin, or another active ingredient hard to dissolve in water.

These are always the focuses of medicine research: improving low solubility, transporting instable or high-toxicity medicine, increasing the amount of the medicine transported to the target tissue, and improving the efficiency of transporting macromolecule medicine into cells. Many of anticancer drugs, anti-AIDS drugs, and immunotherapy drugs are bulky polycyclic compounds of low aqueous solubility and feature hydrophobicity. The hydrophobicity assists these drugs to pass through the lipid bilayer membrane and enter into the cells in some extent and increases the specificity of the drugs to special cell receptors. However, the application thereof usually encounters many difficulties. In oral administration, hydrophobic drugs normally have low absorptivity and poor bioavailability. In intravenous administration, hydrophobic drugs are hard to disperse and likely to block blood vessels and respiratory tracts. Besides, low dispersity also causes the drugs to condense in high concentration, which is likely to induce local toxicity in the body and hinder the drugs from entering blood circulation. Thus, the drugs are hard to absorb and low in bioavailability.

The objective of the present invention is to provide a self-emulsified lipid oil drops as nano-carriers for oral delivery able to effectively transport poorly water-soluble drugs, whereby to overcome the problems encountered in developing hydrophobic drugs. Below, drug-release experiments and animal experiments are used to demonstrate the present invention. In following embodiments but not limit to, curcumin may be used to exemplify the poorly water-soluble drug and verify the bioavailability of the self-emulsified lipid oil drops as nano-carriers.

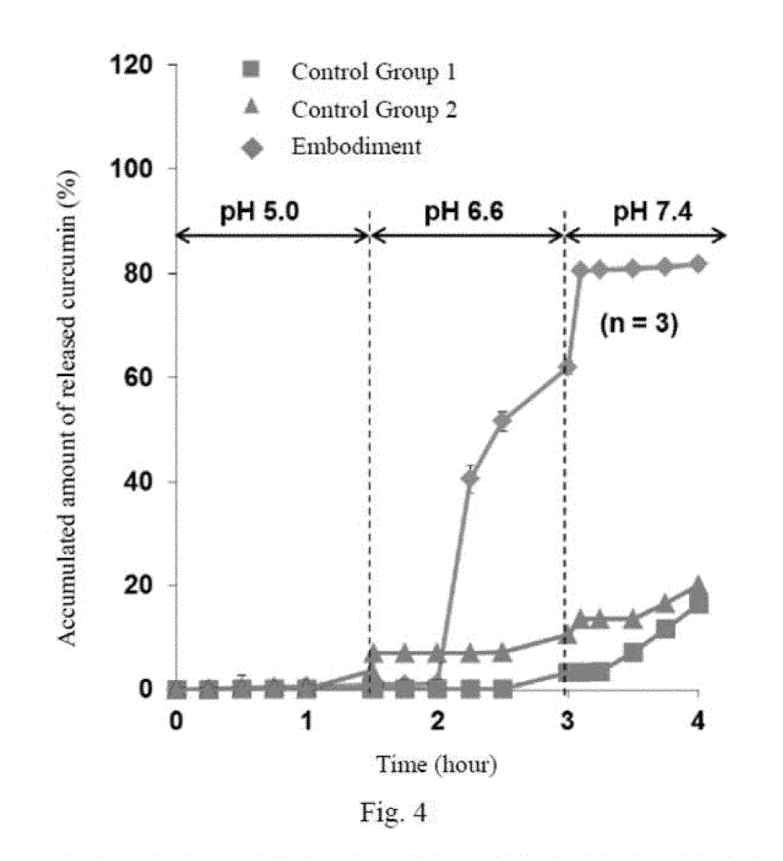

Refer to FIG. 4 for the results of in vitro drug-release experiments for different dosage forms. The embodiment group used in the experiments, but not limited to in the present, adopts the pharmaceutical composition for oral delivery containing curcumin as claimed as the present invention. Control Group 1 uses free-form curcumin without any additive. Control Group 2 uses free-form curcumin with sodium bicarbonate (SBC) added. The compositions of the embodiment group and the control groups are all fabricated into capsules with enteric coating. The capsules of each group is placed in a dialysis bag (MWCO 100 kDa), and the pH buffer, which simulates the physiological environment, is used as the dialysis solution. The dialysis bag is placed and persistently oscillated in an oscillation water bath at a constant temperature of 37° C. The dialysis solution is sampled at specified time points. High-performance liquid chromatography (HPLC) is used to detect the drug released by the bubble carriers in different pH environments. It is observed in FIG. 4: after the experiments have been undertaken for 2 hours, the drug release ratio of the pharmaceutical composition for oral delivery of the present invention is significantly higher than that of the compositions of the control groups. Therefore, the pharmaceutical composition for oral delivery of the present invention is proved to have a very high drug release efficiency.

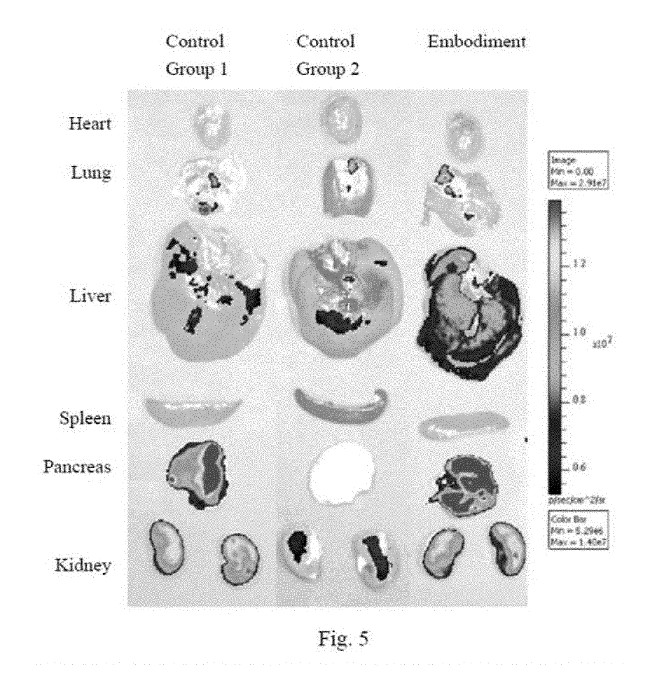

Refer to FIG. 5 showing the distribution of the hydrophobic ingredient of different dosage forms in the tissue of living bodies. Wistar rat (each weighing 300-500 g) are used in the experiments using the in-vivo imaging system (IVIS). In the embodiment of the present invention for the experiments, the curcumin-containing pharmaceutical composition for oral delivery of the present invention is orally delivered with feeding needles to the stomachs of the rat. In Control Group 1, the free-form curcumin is injected hypodermically into the rat. In Control Group 2, the free-form curcumin is orally delivered with feeding needles to the stomachs of the mice. After having taken the drugs for 2 hours, the rat are sacrificed with carbon dioxide. The fresh soft tissues of the rat, including hearts, lungs, livers, spleens, pancreases, and kidneys, are excised, washed, and placed on the imaging bed. Then, the soft tissues are imaged instantly with IVIS. The tissues and bodies of the rat are handled according to the regulations for experimental animals. The primitive data acquired with IVIS is reconstructed and analyzed with the image reconstruction and analysis software to learn the invivo distribution of the multifunctional oral micro particles. In the experiments, the molecular imaging system of IVIS is used to assist in positioning the tissues, and the regions of interest (ROI) of the organs/tissues absorbing drugs are manually selected for quantitative analysis. Thus is acquired the absorptivity of each organ/tissue and the pharmacokinetic distribution of the curcumin-containing compositions.

Refer to FIG. 5, in comparison with Control Group 1 (injecting free-form curcumin hypodermically) and Control Group 2 (delivering free-form curcumin orally), the embodiment of the present invention performs higher absorptivity in livers, pancreases, and kidneys of the rat. Thus, pharmaceutical composition for oral delivery to form the self-emulsified lipid oil drops as nano-carriers of the present invention has good bioavailability.

In conclusion, while exposed to water, the pharmaceutical composition for oral delivery is able to form the self-emulsified lipid oil drops as nano-carriers. The pharmaceutical composition for oral delivery generates monolayer bubble structures containing poorly water-soluble drug that can be converted into double-layer bubble structures containing poorly water-soluble drug near the interface of water and lumen. While the carbon dioxide bubbles of the double-layer nano-assemblies burst at the interface, oil-structured nano-emulsions that contain paclitaxel via self-emulsification can be formed in a living body. The abovementioned bubble structures can effectively transport the poorly water-soluble drug to the recipient organs or tissues of living bodies. Further, the release efficiency of the poorly water-soluble drug of the present invention is higher than that of the conventional dosage form. Therefore, the present invention is highly bioavailable, able to break through the limitation of traditional hydrophobic drugs and provide different directions of drug development.

What is claimed is:

1. A pharmaceutical composition for oral delivery comprising:
a poorly water-soluble drug;
a lipophilic or amphiphilic solvent;
an acid initiator; and
a foaming agent producing carbon dioxide bubbles when the acid initiator is dissolved in the intestinal fluid to form an acidic environment, wherein the poorly water-soluble drug attaches to the solvent to form a self-assembled monolayer carrier system with a bile salts surrounding the carbon dioxide bubbles in water when the pharmaceutical composition is dispersed in an intestinal tract, and lipid oil drops containing the poorly water-soluble drug form when the carbon dioxide bubbles burst at the air-liquid interface in the intestinal tract.

2. The pharmaceutical composition for oral delivery according to claim 1, wherein the solvent comprises capric acid.

3. The pharmaceutical composition for oral delivery according to claim 1, wherein the foaming agent comprises carbonates or bicarbonates.

4. The pharmaceutical composition for oral delivery according to claim 1, wherein the acid initiator comprises organic acid anhydrides or organic acids.

5. The pharmaceutical composition for oral delivery according to claim 4, wherein the acid initiator comprises diethylenetriaminepentaacetic dianhydride (DTPA anhydride), citric acid anhydride, or citric acid.

6. The pharmaceutical composition for oral delivery according to claim 1, wherein the poorly water-soluble drug comprises curcumin, paclitaxel, doxorubicin, or derivatives thereof.

7. The pharmaceutical composition for oral delivery according to claim 1, wherein the pharmaceutical composition for oral delivery is in form of a tablet or a capsule.

8. The pharmaceutical composition for oral delivery according to claim 7 further comprising an enteric coating enveloping the tablet or capsule.

9. The pharmaceutical composition for oral delivery according to claim 8, wherein the enteric coating comprises a methacrylic acid copolymer, hypromellose phthalate, hydroxypropyl cellulose acetate, hydroxypropyl cellulose succinate, or carboxymethyl ethyl cellulose.

10. The pharmaceutical composition for oral delivery according to claim 1, wherein on the condition of a weight of the poorly water-soluble drug in the range of 1-3 milligram, the lipophilic or amphiphilic solvent is in the range of 15-60 milligram, the acid initiator is in the range of 2-25 milligram, and the foaming agent is in the range of 1-20 milligram.
